(19) United States
(12) Patent Application Publication
(10) Pub. No.: US 2010/0247694 A1
(43) Pub. Date: Sep. 30, 2010

(54) COMPOSITION AND METHOD FOR TREAMENT OF INFLAMATION AND INFECTIONS OF THE OUTER EAR CANAL, NOSE AND PARANASAL SINUSES

(76) Inventor: Joseph Di Bartolomeo, Santa Barbara, CA (US)

Correspondence Address: FELIX L. FISCHER, ATTORNEY AT LAW, 1607 MISSION DRIVE, SUITE 204, SOLVANG, CA, 93463, US

(21) Appl. No.: 12/814,259

(22) Filed: Jun. 11, 2010

Related U.S. Application Data
(63) Continuation-in-part of application No. 12/023,926, filed on Jan. 31, 2008.

(51) Int. Cl. A61K 31/375; A61K 31/355; A61K 36/00; A61P 29/00; A61P 31/00; A61P 27/16

(52) U.S. Cl. 424/769; 514/474; 514/458

(57) ABSTRACT

A composition for topical application to the human external ear canal and nasal membranes for relief from symptoms of inflamation and infections and prophylaxis of sexually transmitted diseases the incoporates L-Ascorbic acid in a concentration of about 2% to about 25% wt/vol., within an acidic range or pH level of about 4.0 to 2.0 pH respectively, in an aqueous solution with a pharmaceutically acceptable liquid carrier. The liquid carrier in alternative exemplary embodiments is pure distilled water or normal 0.9% physiologic saline, pH adjusted in a pre determined composition for the desired therapeutic effect. The liquid carrier in alternative exemplary embodiments is pure distilled water or normal 0.9% physiologic saline, depending upon the preferred degree of absorption and bioavailability to the host tissues of the active ingredients in the novel invention. This may include sodium chloride as the preferred Sea Salt—which by analysis contains 99% sodium chloride and calcium, magnesium, sulphate, iron, and copper minerals. Additionally, the composition in alternative embodiment includes a synergistic antioxidant and further incorporates zinc gluconate in certain embodiments. The method for topical application of the composition includes drops, a rinse, cream, gel or saturated wick.

### REFERENCE TO RELATED APPLICATIONS

This application is a continuation in part of U.S. patent application Ser. No. 12/023,926 filed on Jan. 31, 2008 entitled COMPOSITION AND METHOD FOR TREATMENT OF INFLAMATION AND INFECTIONS OF THE GENITALIA, CONTRACEPTIVE AND THE PROPHYLAXIS OF SEXUALLY TRANSMITTED DISEASES having a common inventor with the present application, the disclosure of which is incorporated herein by reference as though fully set forth.

### BACKGROUND

1. Field of the Invention

This invention generally relates to treatment of inflammatory disorders due to microbes and biofilm products in the human body and more particularly to a composition and method which restores the natural pH balance to support the homeorrhesis in the human outer ear canal, nose and paranasal sinuses for synergistic cell division, tissue healing and autoimmune resistance against microbicidal activity while the local acidic milieu for the degradation and inactivation of invading microbes and biofilm products to prevent attachment, penetration and infection by microbes.

2. Description of the Related Art

In man, the mode of transmission of the more common diseases may be extrinsic or intrinsic. Extrinsic causes include the adherence to physical surfaces by environmental microbes, toxins and pollutants, and onto the host tissues. This may be in the form of airborne particles inhaled into the nose and sinus passages. Transmission frequently occurs by physical contact from handshakes, kissing or during sexual activities when the microbes are carried in the secretions or fluids of the body. The intrinsic causes are due to an alkalization of the normal acidic pH milieu of the host tissues and alteration of the composition of the protective surface mantel of the host organ and compromise of the immune defense mechanism of the host.

The mode of interaction is initially by surface contact, followed absorption of the surface nutrients, alteration of the normal commensal flora, colonization by microbes and release of their alkaline or basic pH biofilm and wastes.

The skin is the largest organ of the body. The surface epidermis is essentially a thick dry casing of a scleroprotein, keratin, covering and protecting the internal organs. The surface layer of keratin consists of millions of dead squamous epidermal cells that are completely shed approximately every thirty days. The epidermis is non-permiable and behaves as a lipid barrier. But, once hydrated, the skin becomes more permeable and the barrier is penetrable.

At several regions of the body, the configuration of the skin conforms into pockets, passages or cavities which reach the internal organs of the body, and the substance of the tissue intergrades with additional complementary structures, glands and hair follicles, that maintain the organ homeorrhesis. Exemplary of such specialized organs are the outer ear canal, nose and paranasal sinuses.

This transitional zone of changes in structure and function is seen in the external ear canal, nose and sinuses of humans. These organs, without a keratinized surface layer, instead have semi permeable membranes covered by secretions, both of which are completely regulated by the local milieu of osmolarity, acidity, pH, tissue homeorrhesis, immune system and the nutrients available.

The human ear is divided into three parts: External, middle and inner ear. The external ear only is pertinent to this application. The external ear consists of an auricle and external auditory canal. The auricle (or pinna) is a visible corrugated flap of skin-covered cartilage which projects from the side of the head to collect sound waves. It has an inferior pendulous portion which is free of cartilage, called the lobule. At the lowermost portion of its attachment to the head, the auricle has a scooped-out area called the concha which leads to the opening, or stoma, of the external auditory canal. Anterior to the stoma of the external auditory canal is a small, flat projection of skin called the tragus.

The External Ear Canal is a skin lined, dry, keratinous non permeable, 2.5-3. cm long, tube ending in a cul de sac to form the outer layer of the ultra-thin transparent tympanic membrane in the depth of the canal. There are two points at which the lumen of the external auditory canal is narrow. The superficial one is at the junction of the cartilaginous and bony portions while the second constriction is, deeper and narrower, at a region called the isthmus of the ear canal. The supporting structure of the outer one-third of the ear canal is made up of cartilage, a specialized flexible connective tissue. The inner two-thirds of the ear canal is formed by a ring of bone growing outward from the temporal bone of the cranium.

At the entrance of the ear canal the thick skin (@ 1.5-2 mm) contains an annular grouping of hair follicles and glandular structures. Both the hairs and glandular secretions are easily seen at the entrance of the ear canal. They act as a barrier to foreign matter and produce antimicrobial fluids that block most microorganisms from penetrating the surface and colonizing any damaged or compromised tissues.

In the deeper 1/3 length of the ear canal the skin becomes very thin (@ 0.1-0.15 mm inch thickness), and is devoid of any subepithelial fat or glandular cells. The glands at the entrance include specialized ceruminous glands, sweat glands and sebaceous glands. The glands open at the root or velli of the hair follicle. The ceruminous glands, 1000-2000, are thick sweat glands that release sweat-like liquid. The sebaceous glands produce sebum, an oil-like fluid similar to that produced by similar glands of the skin. Both of these secretions form a biofilm mixture that adheres to the surface of the host cells and flora of microbes.

Fresh cerumen consists of white, watery droplets which, over a period of time, become darkened, thickened, sticky, and semi-solid. Cerumen contains lysozyme, a natural microbicide. The local pH provides a critical acid mantel over the surface that inhibits the overgrowth of opportunist microorganisms in the bacterial flora. The normal pH level of the human ear canal is 4.5 or less, to facilitate the natural homeorrhesis and inhibit the growth of microorganisms.

In the deeper ⅓ of the EAC, the exfoliated layers of skin on the eardrum and the desqumated sheets of keratin (the dead, flattened cells on the outer layer of the skin), here migrate outward to the orifice where they mix with the cerumen, sebum and hairs together forming a product of biofilm. EARWAX contains cerumen, desquamated sheets of keratin, and often includes the sebum, sweat and various foreign substances. These foreign substances can be any exogenous matter that is capable of entering the ear canal: i.e.; insects, soaps, topical creams or cosmetics, sand or other abiotic adhesins.

The ear canal is normally free of any such obstruction for the unimpeded transmission of acoustic energy from environmental sounds. More important, the skin of the ear canal must be dry and to maintain the surface film barrier, acid mantel, preventing penetration by microorganisms and toxins.

For the purposes of this application both cerumen and the collective product earwax are similarly referred to biofilm.

The lining of the nose is a derivative from skin primordium but the epithelium undergoes a dramatic and distinct transition within the organ. In the nasal vestibule, the dry keratinous surface epidermis is replaced by a semi-permeable mucous membrane epithelium lining the passages. Within the entrance at the transitional zone, the sub-epithelial tissues contain a collection of hair follicles and specialized glands situated in an annular formation at the entrance, adapted to guard and protect the distinct host tissues.

The glands in each passage secrete protective fluids that include a microbicide enzyme to keep the unique microbial flora in balance and to protect the host against colonization and infection by overgrowth of surface microbes. The natural liquid product(s) maintain a specific level of acidity, or pH, to support the metabolic need of the local host tissues while being is inconsistent with the survival of microbes.

A disruption of this homeostasis will result in maceration, alkalosis of the liquid biofilm, tissue inflammation, infection and cellular disruption of the tissue defenses. Unless this natural acidic pH is restored, the microbes will adhere to the surface of the host, colonize within the protective biofilm barrier and then pentrate the outer cells of the host where they may infect the target cells, unless they are stopped at the site of contact.

The nasal organ is the body's first line of defense of the respiratory system that filters and removes airborne vapors, particles, pollutants and toxins inhaled. Equally important is the humidification of the inspired air. Normally, the nasal passage is a self-cleansing structure that purifies the inspired air for the delivery of the vital gases to the lungs. Here, exchange occurs through the delicate semi-permeable membranes of the alveoli in the lungs. This nasal respiratory organ then receives the carbon dioxide and gaseous wastes from the lungs to be exhaled from the nostrils back into the environment.

Of special importance is the function of the nasal organ for the removal of particles, air conditioning of the inspired air, and transport of macro-particulate matter. Because of the aerodynamic equivalent diameter (AED). particles of approximately 5 um AED or greater are 85-90% removed by the nose and nasopharynx. Smaller particles penetrate to varying degrees into the lower respiratory tract. Virus-containing droplets coalesce into diameters frequently exceeding 5-6 um and thus are largely retained in the nose.

At the entrance of the nasal vestibule, the keratinizing stratified squamous epithelium abruptly becomes flatter and gradually changes to pseudostratified ciliated columnar as one moves posteriorly.

Most of the posterior two-thirds of the nasal cavity is lined by pseudostratified columnar ciliated epithelium, with goblet cells and mucous and serous glands.

Anteriorly, where there is a paucity of cilia, mucus propagation is slow; it moves only 1 to 2 mm. per hour. Posteriorly, mucus may be propagated 10 mm. each minute and the mucous blanket in the posterior two-thirds of the nose reconstitutes itself every 10 minutes.

Human cilia extend about 6 um above the luminal surface of the cell, and are about 0.3 um in width. Perhaps as many as 100 are the cilium establishes its “beat”. The forward beat is more forceful. Beating occurs 1000 or more times per minute and is metachromous. A mucus blanket covers and diffuses among the cilia. It is a 12-15 um thick, sticky, tenacious, adhesive sheath consisting of two layers, the mucus and the periciliary layers.

In health, the normal intranasal pH level of the mucous is the range of 4 to 7. Its approximate composition includes is mucin, 2.5-3% glycoprotein, 1-2% salts, and 95% water. Immunoglobulins comprise about 70% of the protein content.

The nose and paranasal sinuses secrete more than one quart of thin clear mucous a day, produced by serous and goblet cells, found throughout the nose, sinuses, Eustachian tube, and middle ear. Muramidaze (formerly Lysozyme) and IGA (or secretory immunoglobulin) are also in the mucous blanket. The beating of the underlying cilia propels the blanket of mucus along with trapped macro-particles and dissolved material, in a mostly continuous movement towards the pharyngeal end of the esophagus, where it is swallowed or expectorated.

The mucociliary transport or clearing system is really two systems working simultaneously. It depends on the found on each cell in the nose. The to and fro movement of actively beating cilia which propel the mucus flakes to the upper end of the esophagus and the deeper periciliary fluid also moving posteriorly by the mechanisms poorly understood. The speed of the posterior movement varies widely in apparently healthy individuals from 1-20 mm per minute. Relative mucostasis, usually caused by decreased beat frequency, may permit time for noxious elements to inaugurate disease by penetration of this mucosal barrier.

Boatsman, et al. states that nasal mucociliary clearance is an essential function required to defend and preserve the health of the nose. Between 20 and 40 mL of mucus are secreted from the normal “resting” nose daily from 100 cm-2 of nasal mucosa. This mucus provides a confluent lining for the nasal cavity onto which inhaled particles can impact. Eighty percent of particles larger than 12.5 um are filtered from the air before they reach the pharynx. The blanket of mucus can be moved by the coordinated waves of cilia from the anterior nose to the nasopharynx, where it can be either expectorated or swallowed. Mucociliary transport depends on interactions between the cilia (number, structure, and beating rhythm) and a two-layered system of mucus, 1 micron in thickness, with certain viscoelastic properties. The cilia beat within the lubricating periciliary layer fluid. This layer is anatomically continuous with, but functionally distinct from the outer, more viscous mucus layer. Saccharin, and other soluble materials, can dissolve and be transported within the periciliary layer. Particles may be transported through this layer more effectively than in the layer of mucus above. The nasal mucociliary system is sensitive to drying; however, under usual conditions mucociliary transport rate varies only slightly due to the humidifying and warming functions of the nose.

Homer reported that mucociliary clearance is dependent on a complex series of interactions between mucus, cilia and periciliary fluid but in practice is essentially dependent on ciliary beat frequency and mucus rheological properties. Ciliary beat frequency may be reduced in patients with chronic sinusitis, but the reduction in mucociliary clearance that results is not totally accounted for by this and must occur at least partially to the rheological changes in mucus.

It is therefore desirable to provide an efficacious treatment for the inflammation and infections which occur in the external ear canal, nasal passages and paranasal sinus which accommodates the various symptomatic sources and physical conditions in those organs.

### BIBLIOGRAPHY OF REFERENCES

Boone D, McFarlane S, Von Berg S, The Voice and Voice Therapy, Seventh Edition, New York: Pearson.

Bowie, Andrew G and O'Neill, Luke A. J., Vitamin C Inhibits NF-kB Activation by TNF Via the Activation of p38 Mitogen-activated Protein Kinase, Journal of Immunology, 2000, 165:7180-7188.

Burns, J. J., Missing Step in Man, Monkey and Guinea Pig required for the Biosynthesis of L-Ascorbic Acid. Nature, Letter to the Editor, No. 4585, Sep. 14, 1957: 553.

Dunne, Jr., W. M. FOCUS, Bacterial Adhesion: Seen Any Good Biofilms Lately? Clinical Microbiology Reviews, April 2002, 155-166.

Engler, M., Engler, M., Malloy, M., Chiu, E., Sclotoetter, M., Paul, S., Stuehlinger, M., Lin, K., Cooke, J., Morrow, J., Ridker, P., Rifai, N., Miller, E., Witzum, J., Mietus-Snyder, M. Antioxidant vitamins C and E improve endothelial function in children with hyperlipidemia. Circulation 2003: 108; 1059-1063.

Gallarate, M., Carlotti, M., Trotta, M., Bovo, S. On the stability of ascorbic acid in emulsified systems for topical and cosmetic use. International Journal of Pharmaceutics 1999; 188: 233-241.

Gould Simon W J, Fielder Mark D, Kelly Alison F, Naughton Declan P. Anti-microbial activities of pomegranate rind extracts: enhancement by cupric sulphate against clinical isolates of S. aureus, MRSA and PVL positive CA-MSSA, BMC Complementary and Alternative Medicine 2009, 9:23.

Huisman, Evelien, Uylings H, Hoogland P, Gender-related changes in increase of Dopaminergic Neurons in the Olfactory Bulb of Parkinson's Disease Patients, Movement Disorders, Vol. 23, No. 10, 2008, pp 1407-1413.

Huisman, Evelien, Uylings H, Hoogland P, A 100% Increase of Dopaminergic Cells in the Olfactory Bulb May Explain Hyposmia in Parkinson's Disease, Movement Disorders, Vol. 19, No. 6, 2004.

J. J. Homer, R. J. England, A. D. Wilde, G. R. J. Harwood & N. D. Stafford, The effect of pH of douching solutions on mucociliary clearance, (1999) Clin. Otolaryngol. 24, 312-315.

Landis B, Van H, Guinand N, Horvath J, Haehner A, Savva E, Jugentobler M, Lacroix J, Burkhard P, Retronasal Olfactory Function in Parkinson's Disease, The Laryngoscope, 119: November 2009.

Lee, J., Kim, J., Han, S., Chang, S., Kang, H., Lee, O., Oh, S., Suh, K. The stabilization of L-ascorbic acid in aqueous solution and water-in-oil-in-water double emulsion by controlling pH and electrolyte concentration. J. Cosmet. Sci January/February 2004; 55: 1-12.

Lianou, P. E., Fortis, A. A., Papavassilliou, J. T., The effect of low concentrations of ascorbic acid in microbial adherence in vitro. Int J Vitam Nutr Res. 2003 July; 73 (4): 245-50.

The Maillard Reaction: Chemistry at the Interface of Nutrition, Aging, and Disease

Edited by: John W. Baynes, University of South Carolina, Charleston, S.C.; Vincent M. Monnier, Case Western Reserve University, Cleveland, Ohio; Jennifer M. Ames, University of Reading, Whiteknights, Reading, UK; Suzanne R. Thorpe, University of South Carolina, Columbia, S.C.

Margolis, Sam A., and Park, Edward., Stability of Ascorbic Acid in Solutions Stored in Autosampler Vials, Clinical Chemistry 47, No. 8, 2001, pp 1463-1464. (NIST, Analytical Chemistry Division, Gaithersburg, Md. 20899-8392.

Rawal, B. D. Bactericidal action of ascorbic acid on Pseudomonas aeruginosa-alteration of cell surface as possible mechanism. Chemotherapy (1974), 24, 166-172.

Reed, C., Robinson, D., Lock, E., Antioxidant status of rat nasal cavity. Free radical Biology & Medicine 2003; 34:607-615.

Sienkiewicz-Jarosz H, Scinska A, Kuran W, Ryglewicz D, Rogowski A, Wrobel E, Korkosz A, Kukwa A, Kostowski W, Bienkowski P, Taste responses in patients with Parkinson's Disease, J. Neurol Neurosurg Psychiatry 2005; 76:40-46.

Simpson C, Fleming D, Medical and Vocal History in the Evaluation of Dysphonia, Otolaryngologic Clinics of North America, Vol. 33, Issue 4 (August 2000).

Van Robertson, W. The effect of ascorbic acid deficiency on the collagen concentration of newly induced fibrous tissue. From the Division of Experimental Medicine and the Department of Biochemistry, College of Medicine, University of Vermont, Burlington, Vt., Journal of Biological Chemistry, 1952 May; 196 (1): 403-8.

Van Robertson, W., Schwartz, B. Ascorbic acid and the formation of collagen. From the Division of Experimental Medicine and the Department of Biochemistry, College of Medicine, University of Vermont, Burlington, Journal of Biological Chemistry, 1953 April; 201 (2): 689-696.

Victor M, Ropper A, Principles of Neurology, Seventh Edition, New York, McGraw-Hill.

Westerveld, G., Dekker, I., Voss, H., Bast, A., Scheeren, R Antioxidant levels in the nasal mucosa of patients with chronic sinusitis and healthy controls, Arch Otolaryngol Head Neck Surg 1997; 123 (2): 201-204.

Zhang A, et al, Biofilms and mucosal healing in post-surgical patients with chronic rhinosinusitis, American Journal of Rhinology & Allergy, Volume 23, Number 5, September/October 2009, pp. 506-511 (6).

### SUMMARY OF THE INVENTION

The present invention provides a composition for topical application to the human external ear canal, nose and through the nose, the nasal sinuses for relief from symptoms of inflamation and infections associated with ear canal infections such as otitis externa and otomycosis, sinusitis and rhinitis and to provided a prophylactic effect against recurrence. The composition incorporates L-Ascorbic acid in a concentration of about 2% to about 25% wt/vol., within an acidic range or pH level of about 4.0 to 2.0 pH respectively, in an aqueous solution with a pharmaceutically acceptable liquid carrier.

The liquid carrier in alternative exemplary embodiments is pure distilled water or normal 0.9% physiologic saline, depending upon the preferred degree of absorption and bioavailability to the host tissues of the active ingredients in the novel invention. This may include sodium chloride as the preferred Sea Salt—which by analysis contains 99% sodium chloride and calcium, magnesium, sulphate, iron, and copper minerals.

Additionally, the composition in alternative embodiment includes a synergistic antioxidant such as Tocopherol, water solubilized vitamin E (alpha-tocopherol) in a concentration of about 500 to 1,000 i.u./100 m or a flavone, bioflavonoid, in a concentration of about 0.1 to 0.5% wt/vol. The nasal-sinus composition may employ pomegranate extract in the preferred fraction in the nasal-sinus solution. However, pomegranate extract is preferred only in the composition to treat nasal and sinus symptoms, but is excluded from the preferred composition for the ear canal.

The method for topical application of the composition includes drops, a rinse, cream, gel, or saturated wick.

### DETAILED DESCRIPTION OF THE INVENTION

The isotonic and ionic state of the level of acidity (the pH level) govern the vital biochemical and metabolic processes, extracellular and intracellular, of all living tissues, including microbes. The relative acidity of a solution or tissue is measured according to a pH scale that ranges from 0-14. Those registering above 7 function within an alkaline environment, those below 7 are acidic. A solution with a pH of 7 is considered neutral.

The mathematical symbol pH represents the logarithm of the reciprocal of the hydrogen-ion concentration in gram atoms per liter of solution. The level of the pH regulates the vital biochemical availability of nutrients for the immune system and chemical processes of all tissues, both host and microbes, to survive.

The pH of most drugs act as weak acids or bases that are present in solution as both the non-ionized and ionized species. Non-ionized molecules are usually lipid soluble and can diffuse across the cell membrane. In contrast, the ionized molecules are usually unable to penetrate the lipid membrane because of their low lipid solubility.

An exemplary embodiment of the inventive composition disclosed herein includes L-Ascorbic acid, in the purest form of the mother co-enzyme form. Unfortunately, man is unable synthesize L-Ascorbic acid due to the absence of the biochemical step in the liver of the species to convert L-gulonolactone, which is required for the biosynthesis of L-ascorbic acid. The exemplary embodiment of the composition provides L-Ascorbic acid, (also known as Vitamin C, hexuronic acid, an antisorbutic vitamin) with the chemical formula C6H8O6 and a molecular weight of 176.12, or its derivatives: Ester-C, Ascorbate, L-xyloascorbic acid, 3-oxo-L-gulofuranolactone (enol form), L-3-keto threo hexuronic acid lactone, dehydro ascorbic acid, hydrophobic ascorbic acid, and 2-O-.alpha-D-glocopyranosyl-L-Ascorbic acid, or a suitable derivative in therapeutically effective amounts at the approximate effective pH for a safe and optimal effect in a pharmaceutically acceptable liquid carrier for topical application to the genitalia or anal area to prevent or treat diseases, therein.

This embodiment and the methods described subsequently assure the local availability of the composition directly to the immuno-defense tissues of the external ear canal, nasal passages and paranasal sinuses.

L-ascorbic acid is dissolved in water, the pH of the solution becomes sharply acidic due to the dissociation of the hydrogen ion from the enediol group. The main contribution to the lowering of the pH level is the hydroxy group located on the number 3 carbon (C3).

The disclosed embodiment employs this essential nutrient, L-Ascorbic Acid in the purest therapeutic form and application as a co-enzyme required by man for the production of pre- collagen necessary for the strength of healing tissues. The L-Ascorbic acid or comparable ingredient is in the form of U.S.P. grade, extra pure fine powder, granular powder or pure crystals.

As a pure fine powder, L-Ascorbic acid remains in solution longer than other physical forms. The isotonic composition generates the osmotic gradient to initiates the absorption of the composition. For the exemplary embodiment, L-Ascorbic acid in a concentration of about 2% to about 25% wt/vol., within an acidic range or pH level of about 4.0 to 2.0 pH respectively, is employed. The predetermined low pH of the Embodiment exhibits the most potent microbicidal effect and the deformation of biofilm. The diffusible property modifies the pH of the resulting media to approximate the natural pH for the organ homeostasis.

For a first exemplary embodiment, the liquid carrier for the aqueous solution is provided by pure distilled water. In a second exemplary embodiment. The liquid carrier in alternative exemplary embodiments is pure distilled water or normal 0.9% physiologic saline, depending upon the preferred degree of absorption and bioavailability to the host tissues of the active ingredients in the novel invention. This may include sodium chloride as the preferred Sea Salt—which by analysis contains 99% sodium chloride and calcium, magnesium, sulphate, iron, and copper minerals.

For a first exemplary embodiment to relieve the ear canal symptoms, the liquid carrier for the aqueous solution is provided by pure distilled water. A second exemplary embodiment employed for the preferred relief of nasal and sinus symptoms, the liquid carrier is normal physiologic saline in a pre-determined composition for the desired therapeutic effect described herein. Each exemplary embodiment has been modified for the desired transmembrane absorption and bioavailability of the composition to the host target.

Additionally, an antioxidant such as flavone, bioflavonoid, Tocopherol or water solubilized vitamin E (alpha-tocopherol), is added in alternative embodiments to increase the capillary permeability of the composition. Apha-tocopheral is added in alternative embodiments to increase lipid solubility and enhance the antioxidant properties of the composition. A first exemplary embodiment employs a concentration of about 0.1 to 0.5% wt/vol of flavone. A second exemplary embodiment employs a concentration of about 500 to 1,000 i.u./100 m vitamin E.

An essential metalloenzyme, the trace element, zinc, is added in certain embodiments to facilitate the biochemical recovery of the immune tissues.

The preparation process of the composition is designed to maintain the maximum biochemical therapeutic effect of the composition and method and minimize the Maillard reaction by controlling the parameters of the Composition: its isotonicity, osmolar activity, pH range, temperature, and chemical reaction sequences. The composition may be modified for the synergy of the homeorrhesis of the host organ and tissue immune defenses against encroaching microbes.

The modifications employed in the manufacturing composition are done in accordance with the Maillard principles of maximum solubility under the conditions of manufacturing the embodiment, appropriate and timely temperature controlled and regulated chemical reactions of oxidation and reduction, and predetermined alteration in the hydrogen ion concentration to achieve the preferred pH range for the maximum therapeutic effect, potentiation of antioxidant reactions, with selective inhibition of invading microbes directly, and indirectly, by synergistic support of the host immune system, under the safest condition for the integrity of the living host tissues.

Application of the composition is accomplished topically in several forms according to the present invention.

With respect to issues associated with the external ear canal, adequate aeration of the ear canal is essential to expel the daily secretions and shedding of skin, and elimination of the earwax. Moisture is the enemy of the ear canal skin. Physical obstruction of the ear canal, most often due to excessive earwax, results in retained secretion, hydration and secondary swelling of the skin, especially in the outer ⅔ of the canal. Brief exposure to water during bathing or showering does not cause any disturbance of the tissues of the ear canal, but if the water, foreign liquid or hair shampoo is not expelled, the media becomes stagnant, and the saponified products of hair shampoo or soaps break down the protective film of cerumen. Hydrated skin becomes more permeable.

Excessive or prolonged moisture is the cardinal factor of maceration, and inflammatory diseases of the human ear canal. Microbes and biofilm are attracted to moist surfaces. The most prevalent aquatic dermatosis is a condition known as “swimmer's ear” or external otitis. Prolonged exposure to this contaminated liquid in the ear canal alters the keratin layer of the skin, resulting in maceration (micro cracks), and liquid penetration of the hair follicles and oil gland tubules within the tissues.

Thus, external ear canal infections are five times more prevalent in swimmers than in those who do not swim. This is because of the extended period of exposure to moisture, swimming in posted waterways after rainfall runoff has carried with it sewerage or other animal wastes. Excessive chlorine levels in public swimming pools accelerate keratin degradation.

Repeated use of unclean earplugs returns the altered skin flora and biofilm coating to the compromised the external ear canal. If worn during vigorous manual labor, earplugs close the entrance of the canal, prevent adequate aeration and result in a buildup of perspiration and moisture. The ear canal in Caucasians is narrower and has a more pronounced S-shaped configuration than the ear canal in Asians. Narrow ear canals and a genetic tendency to produce larger amounts of cerumen which can occlude the isthmus of the ear canal increase the retention of moisture and have a greater risk of developing otitis externa.

Inappropriate use of pointed, sharp, or abrasive objects to clean the ear canal (including cotton swabs) only serves to disrupt the integrity of the keratin lining of the canal and open the pores to unrestricted entrance by opportunistic microorganisms residing in the ear canal. Such events lead to excessive itching and pain. Using blunt or sharp objects in an attempt to remove the earwax or biofilm to relieve itching often results in impacting it deeper into the canal bruising the thin skin. Pain increases and the swelling eventually continues to the point of closing the small opening of the canal and increasing the intra-canal pressure.

When these microbes penetrate the acid mantel of the skin, their alkaline by-products increase the pH level to an alkaline range suitable for their colonization suitable for their colonization and penetration of the host tissues. There is a significant correlation between the resulting pH level of the canal and the severity of the otitis externa. The microbes that are most often responsible for diffuse external otitis include bacteria, such as Pseudomonas aeruginosa, Proteus vulgaris, Staphylococcus aureus, or E. coli.

Fungal external otitis (otomycosis) typically follows as a result of invasion by Aspergillus niger or Candida albicans. If furuncles develop, the underlying microorganism is usually S. aureus.

Treatment for the ear canal with the composition defined in the embodiments herein may be accomplished by application topically in the form of ear drops. For the composition defined, two drops administered into the ear canal provides the desired effects.

For the composition defined, 2-4 drops are administered into the ear canal with the effected ear elevated. The tragus is gently compressed into the external ear canal to displace the drops medially, toward the eardrum. The patient remains in that position for 10 seconds at which time a sterile cotton plug is applied to the ear canal. The patient may resume normal activities. After 10 minutes the cotton plug, which was intended to absorb any reverse flow of the ear drops out of the canal that might result in soiling of the garment. The ear canal is left uncovered so that the film of drops may air dry leaving a residue on the ear canal for prolonged therapeutic effect.

Alternatively, the composition may be applied as a spray delivered into the external ear canal. In another alternative, the composition may be delivered in the form of a cream applied to the ear canal. In yet another alternative, the composition may be delivered by a wick saturated with the composition inserted into the ear canal.

Westerveld et al. have reported on the deleterious effects of environmental pollutants and microbial infections on the local immune system and antioxidative defense of the nasal and sinus membranes against inflammation and disease.

Imbalances between oxidant formation and antioxidative defense are associated with the pathogenesis of several chronic inflammatory disorders of the respiratory tract. In the nasal mucosa there are three biologically important antioxidants, reduced glutathione, uric acid and vitamin E.

Decreased levels of both reduced glutathione and uric acid in patients with chronic sinusitis lead to a diminished antioxidant defense, which may be associated with the pathogenesis of upper respiratory disorders. The vitamin E level is less important. One of the functions of uric acid is to prevent the oxidation of Vitamin C and it binds transition metals in forms that will not stimulate free radical reaction. Vitamin C has a major role as an antioxidant in the nasal mucosa, directly, and in conjunction with reduced glutathione (GSH) together for the regeneration of Vitamin E from its radical.

Gould, et. al. has described the synergistic effect of pomegranate and anti-oxidants in the enhanced effect of pomegranate and anti-oxidants on the antimicrobial activities against methicillin-sensitive and resistant Staphylococcus aureus (MSSA, MRSA, respectively) to support host tissue resistance.

For issues associate with the nose and paranasal sinuses, when the nasal membranes cannot provide the moisture to adequately humidify the inspired air, the mucous becomes viscous and produces the well-known symptoms of a “post nasal drip”. If the flow of mucous is impaired, it becomes dehydrated, viscous, stagnant and alkalinized. The air borne particles build up and colonization by microbes occurs in the proteinaceous media of biofilm producing the symptoms of rhinitis, sinusitis or both.

The airborne particles build up by accretion and colonization of the microbes within the proteinaceous media of biofilm related to the symptoms of rhinitis, sinusitis or both.

In a first category classified as respiratory illness sinusitis is one of the most common chronic health problems in the United States, affecting an estimated 34 million people a year, according to the National Institute of Allergies and Infectious Diseases (NIAID). More women are afflicted than men.

Sinusitis is often preceded by rhinitis and rarely occurs without concurrent rhinitis. Rhinosinusitis has a self-reported incidence of 135 per 1,000 of the population per year and was the principle reason for almost 12 million physician office visits during 1995. Sinusitis significantly impacts quality of life measures (e.g., the Medical Outcomes Study SF-36) with decrements in general health perception, vitality and social functioning comparable with that observed in patients who have angina or chronic obstructive pulmonary disease. Sinusitis is one of the main reasons for which an antibiotic is prescribed and for lost productivity in the work force.

The common cold and acute rhinitis, associated with one of the many related rhinoviruses infect persons in all countries and climates, are the most common examples. Infection is believed to occur by the airborne route but transmission can also occur by hand transfer from contaminated surfaces.

Infections with rhinoviruses characteristically occur in the fall (September, October) and spring (April, May) as epidemiologic waves of acute respiratory illness; they also are the cause of a large proportion of summer colds. In persons with a cold, one third of adults and 10-15% of children have had rhinovirus recovered from their nasal secretions

There is no specific treatment directed against propagation of the rhinovirus. Supportive measures are those given under treatment of the common cold. Immunity to reinfection is quite specific and probably lasts only several months to two years after most infections, although detectable serum antibody may persist longer.

Another prevalent virus is the adenoviral, types 1, 2, and 5 which infect virtually all persons early in childhood. Adenoviruses occur in all parts of the world. Man is the principal reservoir of this infection. Transmission is by person-to-person contact. The incubation period may be 3-8 days.

Parainfluenza viral diseases, which are also common, may cause of recurrent sinusitis. Studies by Hilmarsson demonstrated that virucidal activity profiles of fatty alcohols, lipids at a low pH rapidly inactivate viruses such as herpes simplex virus (HSV) and respiratory syncytial virus (RSV) or no activity. But the widespread overuse of antibiotics has led to increased resistance of the microorganisms and the newer higher potency antibiotics have a greater incidence of serious side effects and failure of treatment.

Global symptoms of sinusitis may include headache, facial pressure, Chronic Fatigue Syndrome, and generalized aches. Chronic Fatigue pain is one of the top five symptoms associated with sinusitis.

Recent research on recurrent sinusitis, especially after prolonged use of antibiotics or surgery, has implicated an inflammatory reaction of the membranes due to the presence of chronic opportunistic fungi in the paranasal sinus cavities. Similar fungal sinusitis occurs in human immunodeficiency virus infections (HIV).

Most cases of chronic sinusitis involve fungal elements. Where fungal etiology has been suspected, antifungal agents, such as Amphotericin B, have been used. Fungal Sinusitis may occur following the prolonged use of antibiotics or associated with late stage Human Immunodeficiency (HIV). The common fungi identified include Aspergilous Fusirium Curvularia and others. Occasional colonization will result in a fungus ball (Mycetoma). Systemic treatments included Spornox, Diflucan and possibly Muzorale or Lamosil. A recent form of Amphotericin B, SinuNase by Accentia Pharmaceutical, is being studied as an intranasal spray. But antifungal pharmaceuticals are known for their ototoxicity when absorbed systemically and when administered with certain cardiac drugs.

Post nasal drip and sinusitis, when associated with other systemic disorders such as Parkinsonism, is relieved by treating the nasal component with the embodiment described herein. The symptoms of nasal obstruction, clear discharge or post nasal drip, temporary loss of smell, and fatigue may occur in all of these disorders.

The Parkinson's patient exhibits a hypokinetic dysarthria characterized by reduced loudness, breathy voice, monotony of pitch, intermittent rapid rushes of speech, and soft production of consonants. Duffy (19956) writes that many of these abnormalities can be related to the underlying neuromuscular deficits of rigidity, reduced range of movement, and slowness of movement in the laryngeal muscles. Boone, Daniel R. at page 111. Neurologically, the disturbed basil ganglia and extrapyramidal control circuit results in a hypokinetic dysarthria observed in Parkinson's disease (PD). The symptoms of PD are vastly ameliorated with levodopa, a synthetic dopamine. Id. at page 98.

The presence of nasal, sinus, and upper respiratory symptoms associated with Parkinson's disease has raised a suspicion of drug effects: In particular, phenothiazines, haloperidol, and the neuroleptics pimozide and metroclopramide, used at times as antiemetics. L-dopa is not without significant side effects, including symptoms of rhinitis, sinusitis and vocal dryness. Victor, Maurice at page 1132.

The selective serotonin reuptake inhibitors are uselful in apathetic depressions, but some patients report worsening of the Parkinsonian symptoms. The administration of other dopaminergic agents, such as pergolide, bromocriptine, and nower nonergot preparations, such as ropinirole and pramipexole and to some extent, amantadine. Long-acting preparations of L-dopa may also have similar anticholinergic properties by releasing dopamine from striatal neurons. A primary anticholinergic symptom is dry mouth, and postnasal drip. Anticholinergic agents, one of the most widely used being trihexyphenidyl (Artane) and benztropine mesylate (Cogentin) and amatadine may have toxic effects including dryness of the mouth.

In the striatonigral degenerations and multiple system atrophy disorders, Shy-Drager Syndrome is only one example of dry-mouth symptoms causing dysphonia or even stridor.

Because of the relation between nasal function and oral taste function. More than ⅓ of the patients in the study by Sienkiewicz-Jarosz, H. (page 40) reported subjective smell impairment. Taste problems were indicated by four PD subjects.

PD patients have reported or complained of impaired olfactory function and odor discrimination in an overwhelming majority of idiopathic PD patients. Alternatively, studies have shown that unexplained olfactory impairment in healthy individuals may substantially increase the risk of these subjects to develop PD. Orthonasal tests have scored patients within the range of hyposmia and functional anosmia. Landis, B at page 2280. Landis concluded that retronasal and orthonasal olfactory functions are severely impaired in PD patients, significantly altering the food/intake behavior of PD patients. Additionally, hyposmia is one of the most prevalent symptoms of PD. Huisman, Evelien at page 687.

In an additional category defined as Non-Allergic or Vasomotor Rhinitis, the rhinitis does not depend on the presence of IgE and is not due to an allergic reaction. The symptoms can be triggered by environmental pollutants, cigarette smoke and other pollutants, dry or cold climate, as well as strong odors, alcoholic beverages, and overuse of antihistamines, decongestants or other medications. Other causes may include microbial infections, post surgical scarring or nasal obstruction.

Symptoms of Rhinitis, Sinusitis and Upper Airway Disorders may be Primary in association with local inflammation, infections, allergies, air borne irritants or toxins, chemotherapy or radiation therapy. Secondary symptoms of rhinitis, postnasal drip, or sinusitis may occur as due to rhinitis medicamentosa, adverse drug reactions or side effects of systemic or topical nasal medications, products effects of dry climate or low humidity.

The composition disclosed herein is employed to treat those secondary conditions of rhinitis, sinusitis, postnasal drip and upper respiratory dryness as symptoms that are documented in the adverse drug reaction (also known as rhinitis medicamentosa) associated with the administration of various medications or treatments for these related diseases described herein and further including adverse drug reaction secondary to nasal steroids, pharmaceutical nasal spray, nose drops or sinus rinses. Atrophic rhinitis secondary to infection, surgical procedures or radiation therapy.

According to Robinson, there are significant concentrations of Ascorbate and Alpha-Tocopherol and the activities of Superoxide dismutase (SOD) combined with Cu, Zn and Mn forms, catalase, glutathione (GSH) peroxidase, GSH reductase, and DT-diaphorase in nasal respiratory epithelium (RE), olfactory epithelium (OE). These factors are compromised in stress with reduced Vitamin C levels in the host tissue. The method and composition disclosed herein employs the purist form of L-Ascorbic Acid activated at pH levels below 3.5 at which the antioxidant properties of the invention are greatly enhanced to synergistically degrade the biofilm of the invading micro-organisms to alter the rayologic properties of the mucous blanket and reduce the adherence of these invading microbes to the epithelium of the host of tissues. Reed, C., Robinson, D., Lock, E., at pgs 607-615.

Additionally, Zhang et al reported that with abnormal tissue inflamation, allergic edema, scaring, infection and radiation therapy, immunodefense systems are compromised in the ability to attack or degrade the biofilm resulting from infection, postoperative healing, or radiation therapy. The method and composition disclosed herein includes the purest form of Ascorbic Acid with enhanced properties to reduce or inactivate the invading micro-organisms to break down the resulting biofilm and reduce the adherence of the organisms and their biochemical products to the epithelial lining of the nose, sinus, and upper respiratory tract.

The methods of application of the composition for the desired affect on the nasal passages and paranasal sinuses may be accomplished in a number of forms. The solution of the composition of the embodiments disclosed may be topically administered in the form of drops delivered onto the nasal membranes. In a first alternative, the solution is in the form of a spray delivered onto the nasal sinus membranes. In another alternative, the solution is in the form of a mist delivered onto the nasal sinus membranes. The solution in yet another alternative is in the form of a nebulizer delivered onto the nasal sinus membranes. In yet another alternative, the solution in the form of a rinse is delivered onto the nasal sinus membranes. The solution in the form of a cream is applied to the nasal sinus membranes as another alternative. Finally, the solution in the form of a gel may be applied onto the nasal sinus membranes.

The composition described herein as employed improves the rheologic properties of the mucosal secretions of the upper respiratory tract, restored the natural pH, promotes tissue healing and, improves the immune defense response. Westerveld substantiated these premises in the Scientific Literature. These results effected improved nasal function with relief of the symptoms of rhinitis, post nasal drip, and sinusitis. This preferred composition is employed to restore the tissue health and normal rheologic properties of the mucous blanket overlying the membranes. It is further formulated to increase the bioavailibility of the essential nutrient and chemical process necessary to promote tissue healing and strength., with enhanced anti-microbial activity.

Selected Case Studies and Exemplary Treatment Issues

### Ear:

AW: 37 year-old computer technician has a history of hyperhidrosis and excessive accumulation of wax with intermittent obstruction. He's required adequate aural cleaning of earwax every two months for the past two years. He began on the embodiment described therein. He was taking two drops in each ear canal at bedtime. The ear canal is covered with a sterile piece of cotton for approximately 4-5 minutes to catch any excess wax or eardrops that are released from the ear canal. The cotton was removed and the ear canal was allowed to dry. The production of cerumen within the ear canal diminished markedly. The nature of the dry cerumen the product of dry tenacious ear wax material has changed and the wax is a normal ear wax secretion, less adherent and essentially eliminated more efficiently. The interval between visits for removal of cerumen have been extended for 4-6 months with no episodes of obstruction of the ear canal lumen or interference with his normal hearing

Nose-Sinus

AS: An 80 year-old doctor with chronic sinusitis had difficulty with postnasal drip associated with Parkinson's Disease. He had a buildup of thick mucous. There was secondary affect on the ease of swallowing.

An endoscopic examination of the nose, nasopharynx and hypopharyngeal area was completely normal. Because of the viscous nature of his mucous, he began employing a nasal spray containing the composition of the present embodiments. The preferred therapeutic composition employs the purist form of high solubility L-Ascorbic Acid in physiologic saline with the minerals described herein at a pH of approximately 2.6 to 2.4. The composition may include a Vitamin E, bioflavonoid and pomegranate within the preferred pH. After using two sprays in each nostril twice a day, his symptoms resolved significantly. His nasal drip was unnoticed, and the viscosity of the nasal mucous was observed to be much thinner in accordance with the normal rheologic properties of actual paranasal sinus secretions.

He continues to use the nasal spray form of the embodiment to control normal mucous secretions and postnasal drip which is worse since he developed his Parkinson's Disease.

JD: This 66-year old physician has a ten-year history of chronic rhinosinusitis. Endoscopic sinus surgery was performed. The post-operative course was complicated by an osmia. He continued to have a build-up of thick mucous within the nose and paranasal sinuses because of the absence of the mucoviscous blanket of the nose and the mucociliary transport membranes.

The residual mucous became inspissated during evening time at which caused gagging. Thick plugs of mucous would be expelled during his sleep. He began using the embodiment as described with continued improvement and control of the postnasal drip and accretion of stagnant inspissated mucous. As long as he uses the embodiment, the membranes are moist. The incidence of bacterial infection has been reduced dramatically. His sleep pattern has been improved.

Having now described the invention in detail as required by the patent statutes, those skilled in the art will recognize modifications and substitutions to the specific embodiments disclosed herein. Such modifications are within the scope and intent of the present invention as defined in the following claims.

What is claimed is:

1. A composition for topical application to the external ear canal and nasal membranes for relief from symptoms of inflammation and infections from ear canal infections, sinusitis and rhinitis comprising: L-Ascorbic acid in a concentration of about 2% to about 25% wt/vol., within an acidic range or pH level of about 4.0 to 2.0 pH respectively, in an aqueous solution with a pharmaceutically acceptable liquid carrier.

2. The composition of claim 1 wherein the liquid carrier is pure distilled water.

3. The composition of claim 1 wherein the liquid carrier is normal 0.9% physiologic saline, pH adjusted in a pre determined composition for the desired therapeutic effect

4. The composition of claim 1 in which the L-Ascorbic acid is ascorbate.

5. The composition of claim 1 in which the composition includes a synergistic antioxidant.

6. The composition of claim 5 wherein the antioxidant is Tocopherol.

7. The composition of claim 5 wherein the antioxidant is water solubilized vitamin E (alpha-tocopherol) in a concentration of about 500 to 1,000 i.u./100 m.

8. The composition of claim 5 wherein the antioxidant is a flavone, bioflavonoid, in a concentration of about 0.1 to 0.5% wt/vol.

9. The composition of claim 1 further comprising zinc gluconate and pomegranate extract.

10. A method for treatment of symptoms of inflammation and infections of the external ear canal, nasal passages and paranasal sinuses comprising topical application of L-Ascorbic acid in a concentration of about 2% to about 25% wt/vol., within an acidic range or pH level of about 4.0 to 2.0 pH respectively, in an aqueous solution with a pharmaceutically acceptable liquid carrier.

11. The method of claim 10 wherein topical application is accomplished in a form selected from the set of drops, a rinse, cream, gel, and saturated wick.

12. The method of claim 10 wherein the pH level is selected from between 2.2 pH to about 3.0 pH with a preferred pH range between 2.3 and 2.8 for nasal and sinus respiratory membrane, sinus, and membranes of the upper respiratory tract.

13. The method according to claim 10 in which the solution is applied topically to the external ear canal for the treatment of earwax and infections of the ear canal including otitis externa and otomycosis.

14. The method according to claim 10 in which the solution is applied topically to the nasal sinus membranes to treat post nasal drip, rhinitis or sinusitis that occur due to viral, bacterial or fungal colonization of the nasal passages or sinus cavities.

15. The method according to claim 10 in which the solution is in the form of drops delivered into the external ear canal.

16. The method according to claim 10 in which the solution is in the form of a spray delivered into the external ear canal.

17. The method according to claim 10 in which the solution is in the form of a cream delivered into the ear canal.

18. The method according to claim 10 in which the solution is in the form of a gel delivered into the ear canal.

19. The method according to claim 10 in which the solution is in the form of a saturated wick delivered into the ear canal.

20. The method according to claim 10 in which the solution is in the form of drops delivered onto the nasal membranes.

21. The method according to claim 10 in which the solution is in the form of a spray delivered onto the nasal sinus membranes.

22. The method according to claim 10 in which the solution is in the form of a mist delivered onto the nasal sinus membranes.

23. The method according to claim 10 in which the solution is in the form of a nebulizer delivered onto the nasal sinus membranes.

24. The method according to claim 10 in which the solution in the form of a rinse is delivered onto the nasal sinus membranes.

25. The method according to claim 10 in which the solution in the form of a cream is delivered onto the nasal sinus membranes.

26. The method according to claim 10 in which the solution in the form of a gel is delivered onto the nasal sinus membranes.
